(19) United States
(12) Patent Application Publication
(10) Pub. No.: US 2026/0069317 A1
(43) Pub. Date: Mar. 12, 2026

(54) BONE FIXATION APPARATUS

(71) Applicant: EXVET MEDIKAL DANISMANLIK YAZILIM VETERINERLIK HIZMETLERI SANAYI VE TICARET LIMITED SIRKETI, Ankara (TR)

(72) Inventor: Sencer UYGUR, Ankara (TR)

(73) Assignee: EXVET MEDIKAL DANISMANLIK YAZILIM VETERINERLIK HIZMETLERI SANAYI VE TICARET LIMITED SIRKETI, Ankara (TR)

(21) Appl. No.: 19/105,352

(22) PCT Filed: Sep. 6, 2022
(86) PCT No.: PCT/TR2022/050948

§ 371 Date: Feb. 21, 2025

(30) Foreign Application Priority Data
Aug. 23, 2022 (TR) 2022/013276

(51) Int. Cl. A61B 17/62

(52) U.S. Cl. CPC A61B 17/62

(57) ABSTRACT

A bone fixation apparatus is used in the treatment of bone and joint damage as well as the correction of skeletal deformities with an externally applied device that provides stabilization of damaged limbs. The bone fixation apparatus provides the operator with the speed and ease of application during the procedure, as well as the opportunity to continue the daily life of the applied living thing without any difficulty with the help of its geometric structure.

### CROSS REFERENCE TO THE RELATED APPLICATIONS

This application is the national phase entry of International Application No. PCT/TR2022/050948, filed on Sep. 6, 2022, which is based upon and claims priority to Turkish Patent Application No. 2022/013276, filed on Aug. 23, 2022, the entire contents of which are incorporated herein by reference.

### TECHNICAL FIELD

The present invention relates to a bone detection apparatus that is used in the treatment of bone and joint damage as well as the correction of skeletal deformities with an externally applied device that provides stabilization of damaged limbs.

The bone fixation apparatus provides the operator with the speed and ease of application during the procedure, as well as the opportunity to continue the daily life of the applied living thing without any difficulty with the help of its geometric structure.

### BACKGROUND

Today, there are fixation apparatus used in orthopedic surgery, which is a kind of external fixation used to reshape the limb bones of living beings, which is a limb-sparing technique for treating complex and/or open bone fractures and used in cases of infected bone nonunion that is not compatible with other techniques.

There is the Ilizarov apparatus that is named after the orthopedic surgeon Gavriil Abramovich Ilizarov from the Soviet Union, who pioneered the technique and used in the clinics involved in the state of the art. This apparatus is a circular stabilizer with a modular structure. Stainless steel (or titanium) rings are fixed to the bone with stainless heavy wire (called pins or Kirschner wires). The rings are connected by threaded rods attached with adjustable nuts. The circular structure and tensioned wires of the Ilizarov apparatus provide much more structural support than the traditional one-sided fixation system. This allows for early weight bearing. However, steel nuts are used to fix the pins and rods by tightening in the Ilizarov apparatus. For this reason, the operator has difficulty using these small parts during the application as well as after the application, since these fixing parts are made of steel material, they make it difficult for the living thing to continue its movements in daily life.

In the document numbered US2022175421 related to the subject in the state of the art, a ring configured to surround a broken bone, a holding device placed in a first position on the ring, and a tensioner clamp placed in a second position on the ring. In said document, there is no function that can easily fix the rods and pins on the ring body, and it is insufficient in terms of ease of application. Therefore, it is completely different from our invention.

In the document numbered AU2022204477, which is related to the subject in the state of the art, it is stated as follows; a device includes a plurality of rings. Each ring has a first face, a second face, and at least one slot defined by first and second interior edges of the ring on opposing sides of the slot. The at least one slot penetrates from the first face to the second face. The first face of each ring has a first recess adjacent the slot on the first edge and a second recess adjacent the slot on the second edge. A plurality of posts joins each one of the plurality of rings to an adjacent one of the plurality of rings. The mentioned document does not mention an improvement that can easily fix rods and pins, and it remains insufficient in terms of application efficiency. Therefore, it is completely different from our invention.

In the document numbered CN114176745, which is related to the subject in the state of the art, there is a skeleton external fixing device which includes a fixing frame and a fixing connecting rod assembly connected with the fixing frame, the fixing frame includes a first frame body and a second frame body which are distributed up and down, and the first frame body and the second frame body are connected through the fixing connecting rod assembly; a fixing needle assembly used for fixing the broken bone in a space defined by the first frame body and the second frame body is further arranged in the space in a penetrating mode, and the fixing needle assembly at least includes a third connecting piece oppositely arranged on the first frame in a position-variable mode. First frame is in position variable mode. The third connecting pieces are oppositely arranged on the first frame, the fourth connecting pieces are oppositely arranged on the second frame in a position variable mode, a first fixing needle is arranged between the oppositely arranged third connecting pieces in a penetrating mode, and a second fixing needle is arranged between the oppositely arranged fourth connecting pieces in a penetrating mode. According to the external bone fixing device, the position of the fixing connecting rod assembly or the fixing needle assembly can be conveniently adjusted so as to adapt to different bone fracture positions, and meanwhile a patient can lie in bed to have a rest conveniently. In the mentioned document, there is no mention of an improvement made on the fixing elements and the body, and it is insufficient in terms of application efficiency. Therefore, it is completely different from our invention.

In the document numbered CN209422073, which is related to the subject in the state of the art, an external skeletal fixation needle bar clamp device is disclosed. It relates to the field of medical apparatus and instruments, in particular to a needle clamping device which includes a clamping rod block, a needle clamping block and a connecting screw, the clamping rod block and the needle clamping block are connected together through the connecting screw. The clamping rod block is provided with a supporting rod clamping hole and a clamping rod elastic hole, and the connecting portion of the clamping rod elastic hole and the supporting rod clamping hole is arranged to be of an open structure. The needle clamping block is provided with a bone needle clamping hole and a needle clamping elastic hole, and the connecting portion of the bone needle clamping hole and the needle clamping elastic hole is arranged to be of an opening structure. The needle rod clamping device is simple in structure, reasonable in design, simple and convenient in processing procedures of all parts, and large in clamping force of the bone needle clamping hole and the supporting rod clamping hole, so that the bone needle and the supporting rod are prevented from displacing, and the recovery effect is effectively guaranteed. In said document, there is an external clamp developed for fixation, but its technical features are completely different from our present invention and any improvement on the ring body is not mentioned.

The document numbered CN208525003, which is related to the subject in the state of the art belongs to the technical field of medical equipment's technique and specifically relates to an external fixation frame and fracture treatment equipment is related to, this external fixation frame includes: a plurality of fixed modules, a plurality of fixed modules can be dismantled the connection in proper order and can enclose and circularize the structure. The fixed module includes two fixed plates and an at least connecting rod, two fixed plates pass through the connecting rod and connect, the fixed plate includes the first side wall, the first side wall with treat that fixed skeleton is relative, be provided with a plurality of locking through-holes at the first side wall, a plurality of locking through-holes have at least one to be connected with and to be used for the fixed skeletal locking needle spare of treating.

In said document, modular fixing apparatuses are mentioned, but an improvement in connection and fixing elements for ease of application is not mentioned. Therefore, it is completely different from our invention.

The document numbered CN106361420, which is related to the subject in the state of the art relates to an external skeletal fixation orthopedic frame, which includes a near end ring, a far end ring, a connecting sheet and a taylor rod, wherein each of the near end ring and the far end ring is a metal ring with round holes; the round holes are uniformly distributed in the ring; the connecting sheet is fastened on the round holes of the near end ring or the far end ring; the taylor rod includes a stud, a sleeve and a universal joint; the stud is telescopically arranged in the sleeve through screw threads; the elongation or the shortening of a connecting rod is realized through helical transmission of the stud; the lower end of the sleeve is hinged to the connecting sheet or the far end ring through the universal joint; the upper end of the stud is hinged to the connecting sheet or the near end ring through the universal joint. The external skeletal fixation orthopedic frame is improved and designed on the Taylor frame technology; the external skeletal fixation orthopedic frame which can improve the surgical safety and the flexibility and provides a larger skeletal orthopedic space is provided. In the mentioned document, there is no mention of an improvement made on the fixing elements and the body, and it is insufficient in terms of application efficiency. Therefore, it is completely different from our invention.

The document numbered CN105167827, which is related to the subject in the state of the art discloses an external skeletal fixation device which is used for bone fixation. The external skeletal fixation device includes bone needles and at least one mount body. The mount body is provided with a plurality of through holes. The bone needles match the through holes. Two ends of each bone needle are provided with connecting parts. One end, which passes through the through hole, of the bone needle body is directly fixed and connected with the bone through the connecting part, and the other end is fixedly connected with the mount body through the connecting part. The length of the mount body matches the width of the transverse diameter of the bone. The mentioned document does not mention an improvement that can easily fix rods and pins, and it remains insufficient in terms of application efficiency. Therefore, it is completely different from our invention.

The document numbered CN104323842, which is related to the subject in the state of the art relates to an external skeletal fixator, which includes a transverse support frame, an external fixator and a longitudinal fixator, wherein the longitudinal fixator includes an outer frame and a bone surface fixing assembly, the bone surface fixing assembly includes a plurality of fixing blocks. The fixing cables are arranged among the fixing blocks, the fixing blocks are fixedly arranged on the outer frame, and a support framework is defined among the fixing cables through the fixing blocks. It is mentioned that the external skeletal fixator has the advantages that the matching of the outer frame and tensioning wires is adopted, one fixing mesh or a plurality of fixing meshes is/are formed through the tensioning wires.

In said document, a method that makes the rod and pin tightening process easier for the operator during the application is not mentioned and it is completely different from our invention.

The document numbered CN104323836, which is related to the subject in the state of the art relates to a joint part external skeletal fixator, which includes an external fixator assembly and a joint fixator, wherein the external fixator assembly is fixedly arranged on the limb part of the body, the joint fixator includes an outer framework and a plurality of bone surface fixation assemblies arranged on the outer framework, the outer framework is fixedly arranged on the outer part of the body through the external fixator assembly, the outer framework includes at least two fixing frames, each bone surface fixing assembly includes a plurality of fixing blocks, fixing cables are arranged among the fixing blocks, the fixing blocks are fixedly arranged on the fixing frames, a support framework formed by at least two fixing cables is arranged between the fixing frames, and the support framework is in contact with the bone surface and realizes the support on the bone. In said document, there is no mention of an improvement made on the fixing elements and the body, and it is insufficient in terms of application efficiency. Therefore, it is completely different from our invention.

In both current applications and in the document referred to above, the outer frame, which is formed by the use of fixing apparatus and supporting the broken body parts of living things, to fix the broken bone, while forming the outer frame, the nuts used to fix the materials such as rods and pins are small, thus this makes the work of the operator difficult during the application, causes the application to take a lot of time and energy due to the fact that these elements are made of steel and are used in large numbers on the outer frame, it causes the weight to increase, thus restricting the movements of the living thing to which the application is made.

As mentioned above, the tools and apparatus used to fix the broken bones currently used do not provide convenience and time savings to the operator during the application, and do not provide efficiency by restricting the movements of the treated living thing after the application.

### SUMMARY

Bone fixation apparatus of the present invention includes a body, which is made of aluminum material and has a geometric structure in more than one different shape in accordance with the anatomy of the bone to which the application is made, a rod hole on the body, through which the rods required to construct the outer frame are passed and characterized in that the present invention includes a rod fixing slot, which is in line with the rod hole on the side surface of the body, and which provides fixing the rod by tightening it by inserting a setscrew, a pin hole, located on the side surface of the body, next to the rod fixing slot, through which the pin is passed through the bone of the living thing to which the application is made, a pin fixing slot on the body, which is in line with the pin hole, has an internal threaded structure, and provides fixing the pin by tightening it by putting a setscrew.

### BRIEF DESCRIPTION OF THE DRAWINGS

Bone fixation apparatus is realized to fulfill the aims of the present invention as shown in the figures.

In these figures;

FIG. 1 is a perspective view of the 360 degree ring version of the inventive bone fixation apparatus.

FIG. 2 is a top view of the 360 degree ring version of the inventive bone fixation apparatus.

FIG. 3 is a side view of the 360 degree ring version of the inventive bone fixation apparatus.

FIG. 4 is a side view of the 270 degree ring version of the inventive bone fixation apparatus.

FIG. 5 is a side view of the 180 degree ring version of the inventive bone fixation apparatus.

FIG. 6 is a side view of the 52 degree ring version of the inventive bone fixation apparatus.

FIG. 7 is a perspective view of the version of the inventive bone fixation apparatus applied to the jawbone.

FIG. 8 is a perspective view of the version of the inventive bone fixation apparatus applied to the tibia.

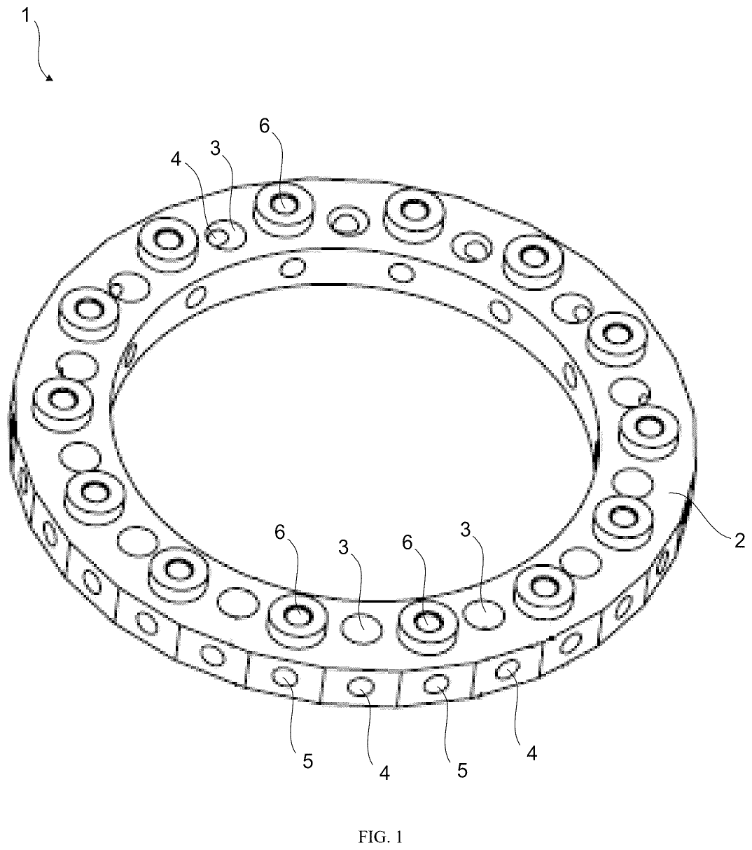

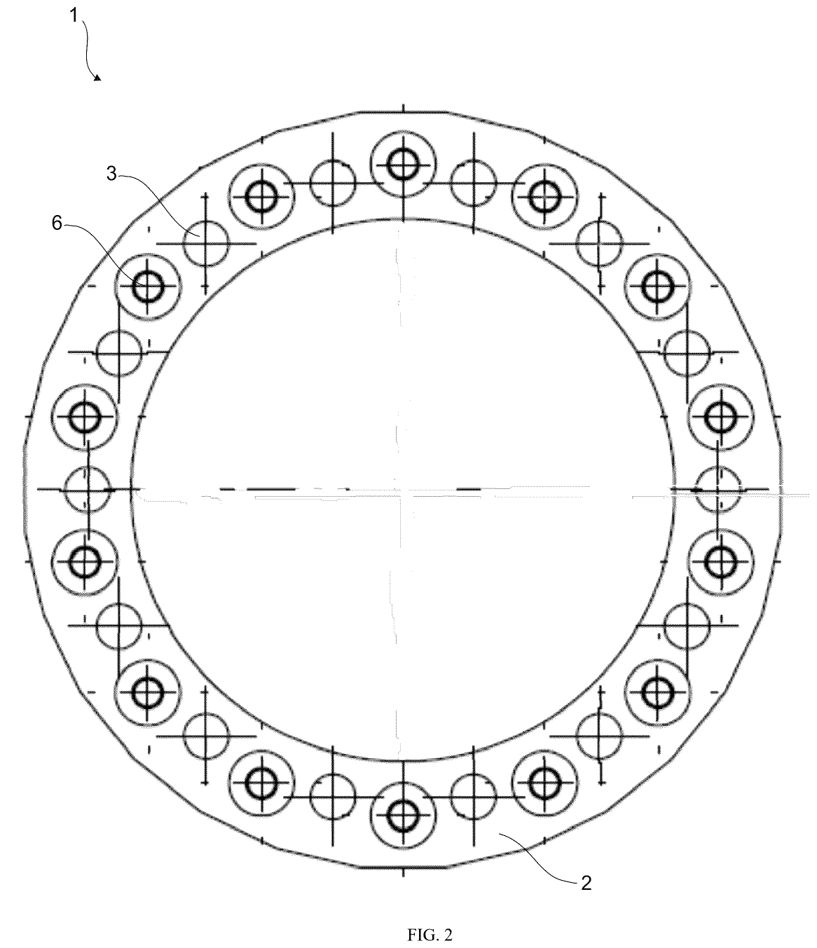

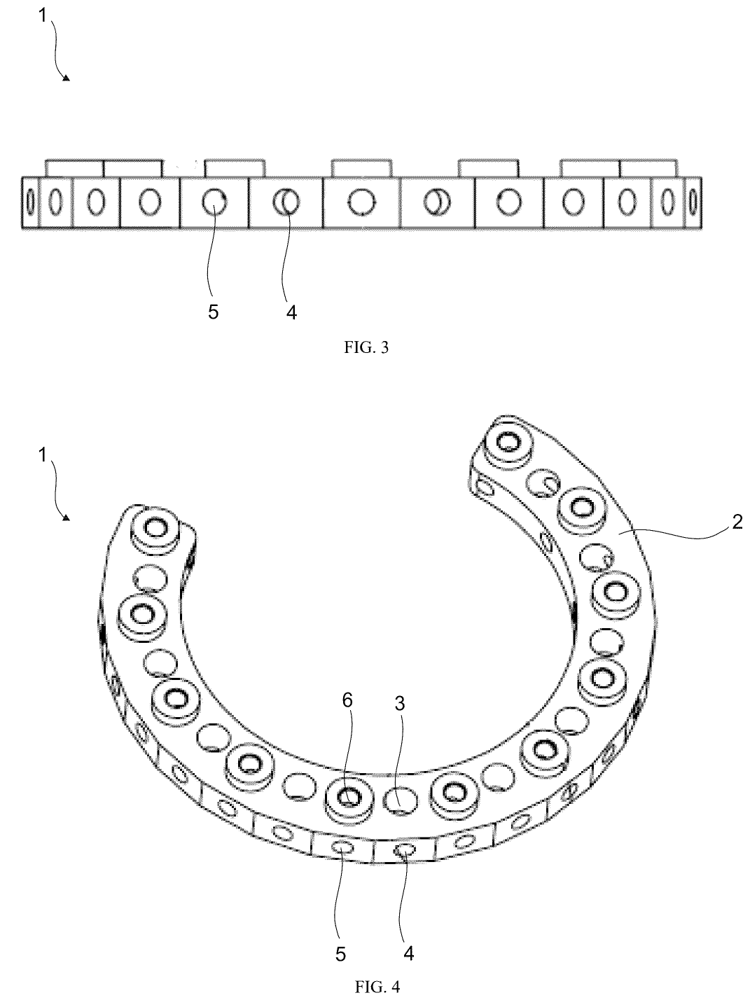

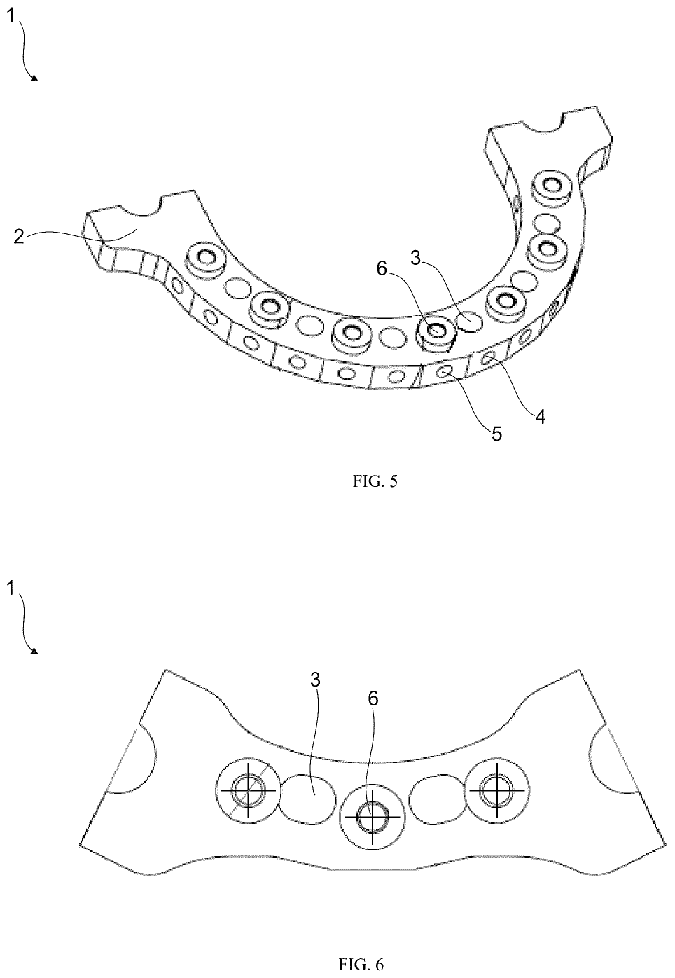

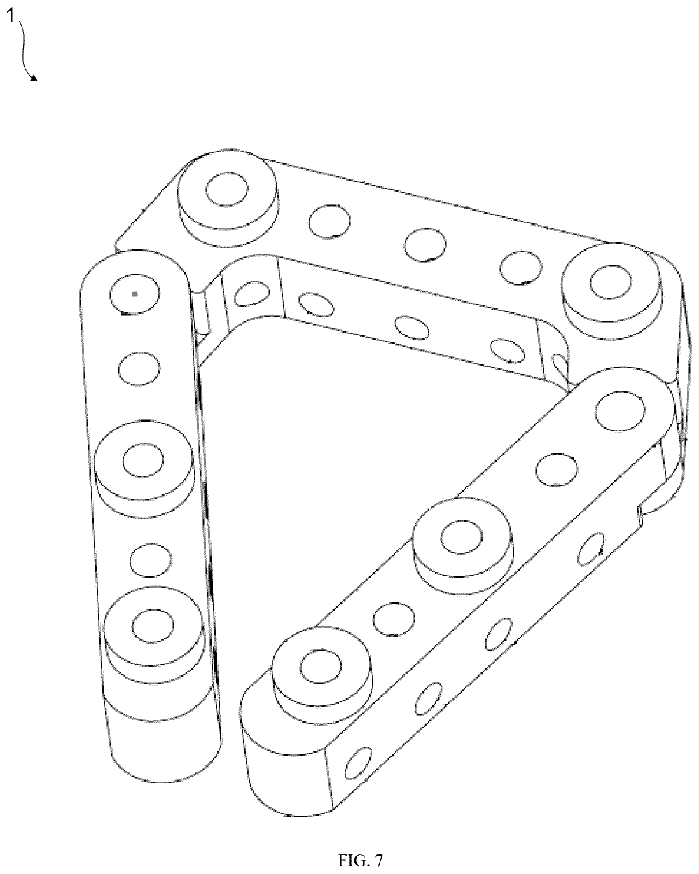

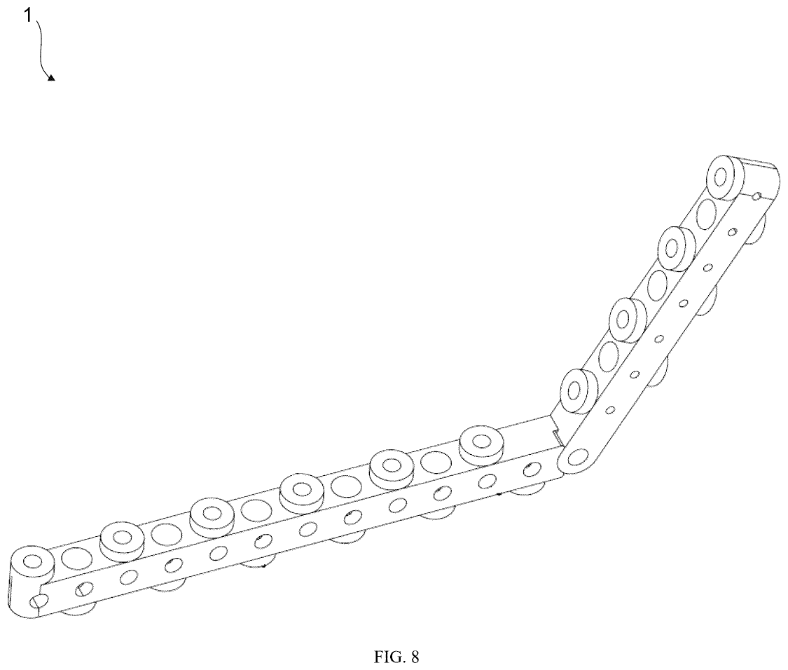

The parts that constitute the inventive bone detection apparatus are enumerated as follows in the attached figures:
1—Bone Fixation Apparatus
2—Body
3—Rod Hole
4. Rod Fixing Slot
5—Pin Hole
6—Pin Fixing Slot

The inventive bone fixation apparatus (1) includes of the following;
Body (2), which is made of aluminum material and has a geometric structure in more than one different shape in accordance with the anatomy of the bone to which the application is made,
Rod hole (3) on the body (2), through which the rods required to construct the outer frame are passed,
Rod fixing slot (4), which is in line with the rod hole (3) on the side surface of the body (2), and which provides fixing the rod by tightening it by inserting a setscrew,
Pin hole (5), located on the side surface of the body (2), next to the rod fixing slot (4), through which the pin is passed through the bone of the living thing to which the application is made,
Pin fixing slot (6) on the body (2), which is in line with the pin hole (5) has an internal threaded structure, and provides fixing the pin by tightening it by putting a setscrew.

It provides both ease of application and time savings to the operator with the help of the rod fixation slot (4) and the pin fixation slot (6) of the body (2) of the inventive bone fixation apparatus (1) during the construction of the outer frame on the applied bone, during the fixing of the rods passed through the rod hole (3) and the pins passed through the pin hole (5).

The rods passed through the rod hole (3) and the pins passed through the pin hole (5) can be easily fixed with the help of a setscrew due to the fact that the rod fixing slot (4) and the pin fixing slot (6) are internally threaded.

The weight does not increase much when the outer frame is formed, since it does not require an external connection piece such as a nut and pin holder for fixing with the help of the geometric structure of the rod fixing slot (4) and the pin fixing slot (6). In this way, the living thing to which the application is made can easily continue its daily life without any difficulties.

Rod fixing slot (4) and pin hole (5) are located on the side surface of the body (2) of the bone fixation apparatus (1), alternately along the body (2). Likewise, the rod hole (3) and the pin fixing slot are located on the upper surface of the body (2) of the bone fixation apparatus (1), along the body (2), respectively.

The body (2) of the inventive bone fixation apparatus (1) to be applied to different types of animal bones can be adopted as; 52 degree ring, 180 degree ring, 270 degree ring, 360 degree ring, it can be adapted as two bars of varying sizes, after being joined at different angles, and fixed with a bolt to be applied in the tibia and it can be adapted to be used in different areas, by combining three bars of varying sizes from two points, so that they can be opened and closed thanks to its movable mechanism to be applied to different types of animal jawbones.

What is claimed is:

1. A bone fixation apparatus, comprising:
a body, wherein the body is made of aluminum material and has a geometric structure in more than one different shape in accordance with an anatomy of a bone to which an application is made,
a rod hole on the body, wherein a rod required to construct an outer frame is passed through the rod hole,
a rod fixing slot, wherein the rod fixing slot is in line with the rod hole on a side surface of the body, and provides fixing the rod by tightening the rod by inserting a first setscrew,
a pin hole, wherein the pin hole is located on the side surface of the body, next to the rod fixing slot, and a pin is passed through the pin hole through the bone of a living thing to which the application is made, and
a pin fixing slot on the body, wherein the pin fixing slot is in line with the pin hole, has an internal threaded structure, and provides fixing the pin by tightening the pin by putting a second setscrew.
